(19) United States
(12) Patent Application Publication
(10) Pub. No.: US 2008/0244972 A1
(43) Pub. Date: Oct. 9, 2008

(54) Method for Converting Hyrogenous Gaseous Flows Arising From Chemical Reactor Units Using Hydrogen

(76) Inventors: Pascal Tromeur, Cercottes (FR); Francois-Xavier Haulle, Paris (FR)

Correspondence Address: AIR LIQUIDE;Intellectual Property, 2700 POST OAK BOULEVARD, SUITE 1800, HOUSTON, TX, 77056, US

(21) Appl. No.: 10/576,829

(22) PCT Filed: Oct. 19, 2004
(86) PCT No.: PCT/FR2004/050511

§ 371 Date: Mar. 5, 2007

(30) Foreign Application Priority Data
Oct. 31, 2003 (FR) 0312819

(51) Int. Cl. C01B 3/50; C01B 3/02; B01D 53/047; C07C 4/06

(52) U.S. Cl. 48/127.1; 95/8; 95/96; 423/648.1

(57) ABSTRACT

The invention relates to a method for converting gaseous effluents based on hydrogen arising from at least two reactor units R1 and R2 consuming hydrogen. Said effluents have differing degrees of hydrogen purity. The different hydrogenous effluents are treated in a gas separation unit U for said different hydrogenous effluents, whereupon highly pure hydrogen can be obtained and can be used to feed an additional reactor unit R3. The unit U also produces a residual flow having a low degree of hydrogen purity which can be sent to the combustible gas network of the petrochemical installation.

The present invention relates to a process for recovering in value hydrogen-based effluents resulting from chemical reaction units employing hydrogen.

Numerous petrochemical processes employ a hydrogenation stage in which hydrogen-rich gases are used. This is the case of the synthesis of building-block chemicals combining products of high chemical activity resulting directly from petroleum hydrocarbons. The main building-block chemicals involving significant hydrogen-rich gases are as follows:
ammonia, methanol,
aromatic hydrocarbons: benzene, toluene, xylenes,
cyclohexane, aniline, toluenediamine (TDA),
adipic acid, hexamethylenediamine (HMDA), caprolactam,
oxo alcohols, butanediol (BDO), hydrogen peroxide,
chlorine, styrene, linear alkylbenzenes (LAB), methyl ethyl ketone (MEK).

The processes for the synthesis of these building-block chemicals have at least three characteristics in common. First of all, they all employ a hydrogenation stage during the synthesis of the building-block chemical. Subsequently, these processes all use the recycling of a gas rich in hydrogen (from 50% to 95% by volume). Finally, these processes carry out a partial bleeding of their loop for recycling the hydrogen-rich gas, so as to limit the accumulation of inert materials in this loop. The hydrogen consumed chemically or lost by mechanical losses, dissolution or bleeding is compensated for by a hydrogen-rich makeup gas, the composition of which varies according to its method of production. Although the operating conditions and the compounds treated vary according to the processes, it is generally recorded:
that the total consumption of hydrogen is high in comparison with the weight of product to be hydrogenated,
that the hydrogen/hydrocarbon ratio is much greater than the amount of hydrogen theoretically necessary for the reaction,
that the makeup hydrogen has to be of high purity in order to limit the losses of hydrogen by bleeding.

Thus, it is necessary, during these various processes, to resort not only to a makeup hydrogen of high purity but also to the removal of a gas which is still rich in hydrogen by bleeding the recycling loop. In many industrial cases, the performances of the petrochemical units and in particular the grades of the products obtained are limited by the purity of the feed hydrogen. Furthermore, under the effect of the bleeding operations carried out on recycle gas, the consumption of hydrogen increases with the reduction in purity of the makeup gas; this results in an additional operating cost for the unit.

In order to avoid these problems, the proposal has been made to increase the hydrogen partial pressure in the reaction region. A first solution consists in bleeding off a fraction of the recycle gas in order to limit the concentrations of inert materials (light hydrocarbons, traces of reactants or of hydrogenated product, and the like) therein. However, there are a number of disadvantages to this high-pressure bleeding operation:
The impact on the hydrogen partial pressure is generally fairly low.
As the recycle gas is rich in hydrogen, one of the consequences of the bleeding operation is a loss of hydrogen towards the fuel gas system. This hydrogen is then recovered in value to a slight extent as fuel gas.
Due to this loss, a larger amount of makeup gas has to be introduced.

A second solution consists in purifying the gas from the recycling loop by the adsorption technology of PSA type. This method is only used to a certain extent in petrochemistry as the only gas treated is of moderate purity (70 to 90% by volume H₂) and the yields obtained for a purity of greater than 99% by volume are mediocre. Consequently, the loss in hydrogen brought about renders adsorption not very attractive.

A third solution, such as disclosed in U.S. Pat. No. 6,179,966, consists in treating the hydrogen-rich effluent with a reverse selectivity membrane. The advantage of this type of membrane, in comparison with hydrogen-selective membranes, is that of keeping the hydrogen under pressure. On the other hand, a compromise has to be found between the hydrogen purity desired and the yield. Thus, the gaseous effluent from the hydrogenation of benzene to give cyclohexane can be treated in order to change from a hydrogen purity of 75% by volume to 90%, with the condition of losing approximately 30% of the hydrogen treated. Likewise, in the case of the hydrogenation of nitrobenzene to give aniline, the change from a hydrogen purity of 83% by volume to a hydrogen purity of 95% results in a loss of nearly 40% of the hydrogen treated.

The purity of the hydrogen of the makeup gas required by each petrochemical process is generally greater than 99% by volume. In point of fact, there is not always a source of hydrogen available close to the process (catalytic reforming, steam cracking, and the like) and the hydrogen has to be supplied at the required purity by dedicated producers. If a source of hydrogen is available nearby, the hydrogen produced is purified in order to meet the specifications of the makeup gas. The techniques for purifying the makeup gas are then similar to those mentioned above for the recycle gas. The two principle routes for purifying a hydrogen-rich gas remain adsorption (PSA) or cryogenic separation, followed by a methanation stage, since the compounds CO and CO₂ are poisons for most of the hydrogenation catalysts used in petrochemistry.

One aim of the present invention is to solve the above problems and more particularly to reduce the overall hydrogen consumption of petrochemical processes employing a hydrogenation stage.

Another aim is to debottleneck or also increase the treatment capacity of certain petrochemical processes employing a hydrogenation stage by purifying the main makeup gas and/or by recovering the hydrogen molecules lost in the bleeding operations.

The characteristics and advantages of the invention will become apparent on reading the description which will follow. Embodiments of the invention are given as nonlimiting examples illustrated by the appended drawings, in which:

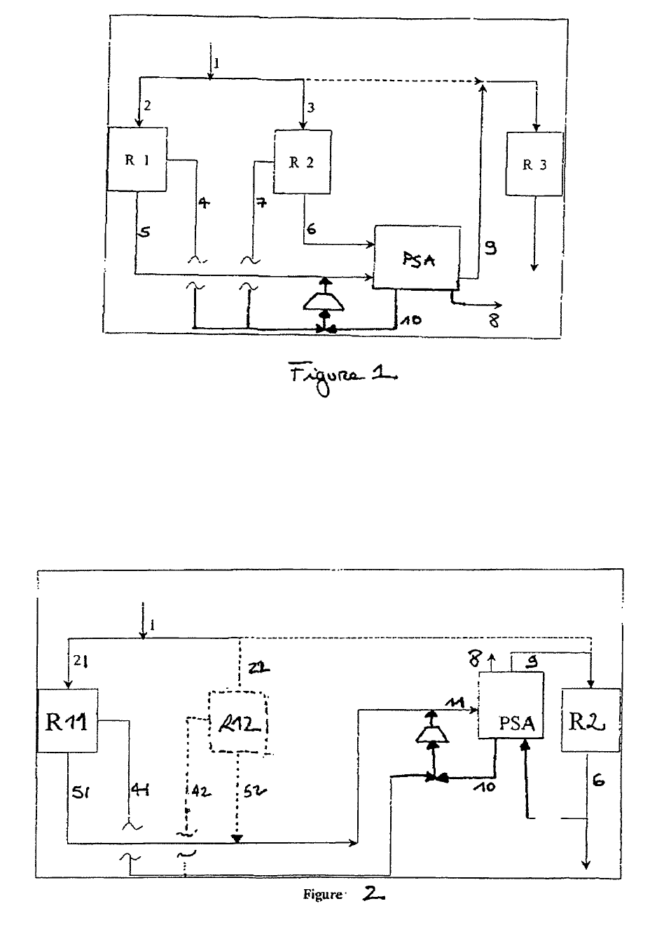

FIG. 1 is a scheme of the invention,

FIG. 2 is a scheme of an alternative form of the invention.

With these aims, the invention relates to a process for recovering in value hydrogen-based gaseous effluents resulting from at least two reaction units R1 and R2 in which hydrogen is consumed, the unit R2 producing a hydrogen-rich gaseous effluent at a pressure P and optionally a hydrogen-poor gaseous effluent and the unit R1 producing at least one hydrogen-poor gaseous effluent, in which process the following stages are carried out:
during stage a), all the hydrogen-poor gaseous effluents resulting from R1 and optionally from R2 are mixed so that the mixture obtained exhibits a pressure P,
during stage b), the mixture of all the hydrogen-poor gaseous effluents resulting from R1 and optionally from R2, adjusted to the pressure P during stage a), is treated in a gas separation unit U fed with the hydrogen-rich gaseous effluent resulting from the unit R2 so as to provide, at a first outlet, an enriched stream exhibiting a greater hydrogen concentration than that of the hydrogen-rich gaseous effluent resulting from the unit R2 and, at a second outlet, a waste stream,
during stage c), the enriched stream resulting from the first outlet of the unit U is reinjected into a reaction unit R3 in which hydrogen is consumed.

The invention consists of the installation of a gas separation unit U between the hydrogen gas systems of several reaction units in which hydrogen is used, such as units of a petrochemical site. The gas separation unit U treats the hydrogen-comprising gases of different hydrogen purities from the units R1 and R2 in order to feed the reaction unit R3 with high-purity hydrogen or in order to purify the recycle hydrogen from this reaction unit R3 without loss in yield. The invention makes it possible to achieve the objectives set by use of the hydrogen-comprising effluents from several reaction units in which hydrogen is consumed and in particular at least two reaction units R1 and R2. The term “reaction unit” is understood to mean a production site on which a reaction is carried out; the reaction unit can equally well be a reactor as a combination of tanks in which the various effluents from a production operation are collected. These units have to be chosen so that their effluents exhibit certain characteristics, it being understood that, according to the process of the invention, it is possible to treat only a portion of each of the effluents. The two units R1 and R2 each have to produce at least one effluent comprising hydrogen at different concentrations. The unit R2 produces a hydrogen-comprising effluent exhibiting a greater hydrogen concentration than all the other hydrogen-comprising effluents resulting from the units R1 and R2. Thus, the term “hydrogen-rich effluent” denotes the effluent resulting from the reaction units which exhibits the greatest hydrogen purity. Generally, this hydrogen-rich effluent exhibits a hydrogen concentration of between 50 and 99% by volume. This hydrogen-rich effluent resulting from the unit R2 can exhibit a pressure P of at least 5 bar, preferably of at least 15 bar. The other gaseous effluents resulting from the units R1 and R2 are “hydrogen-poor”, which means that, for each one, the value of their hydrogen concentration is lower by at least 10% than the value of the hydrogen concentration of the hydrogen-rich effluent, preferably lower by at least 15% and more preferably still lower by 15 to 50%. According to the invention, R2 produces at least one hydrogen-poor gaseous effluent; preferably, this hydrogen-poor gaseous effluent exhibits a pressure close to the pressure P. According to the process of the invention, other additional hydrogen-poor gaseous effluents can be produced, either by R1 or by R2. According to the invention, the pressure of the hydrogen-poor effluent or of the mixture of the hydrogen-poor effluents is adjusted so that it is close to P. If only R1 produces a hydrogen-poor effluent, the pressure can be adjusted by compression or loss of head. If R1 and/or R2 produce several hydrogen-poor effluents, they are all mixed so that their mixture exhibits a pressure equal to P. In order to obtain such a pressure, it may be necessary to compress a portion of the hydrogen-poor effluents with a pressure less than the pressure P. However, this compressing may be optional if at least one of the hydrogen-poor effluents exhibits a pressure greater than P. In addition, if the pressure of one of the hydrogen-poor effluents is greater than P, it is possible to reduce its pressure, for example by loss of head means. The invention also covers the process where the hydrogen-poor effluent resulting from R2 or the mixture of the hydrogen-poor effluents resulting from R1 and optionally from R2 already exhibits a pressure close to P; in these cases, no adjustment of pressure is necessary. By treatment of these various effluents, the invention makes it possible to enrich the hydrogen-rich effluent resulting from R2 so as to be able to use it in a reaction unit in which hydrogen is consumed. This enriching is obtained by depleting the hydrogen-poor effluents. The unit thus produces the enriched stream, generally exhibiting a hydrogen purity of greater than 99% by volume, and the unit also produces a waste stream of low hydrogen purity and of low pressure which can be conveyed to a fuel gas system. The pressure and the hydrogen concentration of the waste stream are respectively less than the pressure and hydrogen concentration values of all the effluents entering the unit U.

According to a specific alternative form of the invention, the reaction unit R3 in which hydrogen is consumed can be the reaction unit R2. In this case, the invention makes it possible both to recover the hydrogen-rich gaseous effluent resulting from the reaction unit R2 and to enrich it in hydrogen using the hydrogen-poor gaseous effluent resulting from R1 so as to be able to recycle this enriched effluent in the unit R2. During this alternative form, it may be necessary for the hydrogen-rich gaseous effluent produced by the unit R3 (or R2) to be compressed before feeding the gas separation unit (U).

According to the invention, the gas separation unit (U) is preferably of the adsorption type. Preferably, the gas separation unit (U) is a pressure swing adsorption (PSA) unit in combination with an incorporated compressor in which use is made, for each adsorber of the unit, of a pressure swing cycle comprising a sequence of phases which define adsorption, depressurization, purge and repressurization phases, such that:
during the adsorption phase:
   during a first stage, the hydrogen-rich gaseous effluent exhibiting a pressure P resulting from the unit R2 is brought into contact with the bed of the adsorber, and
   during a second stage, the mixture with a pressure P composed:
      on the one hand, of the mixture of all the hydrogen-poor gaseous effluents resulting from R1 and optionally from R2 adjusted to the pressure P during stage a),
      on the other hand, of the recycle gas from the PSA,
      is introduced into contact with the bed of the adsorber,
   so as to adsorb the compounds other than hydrogen and to produce, at the head of the bed of the adsorber, the enriched stream exhibiting a greater hydrogen concentration than that of the hydrogen-rich gaseous effluent resulting from the unit R2,
during the depressurization phase, the waste stream from the PSA is produced,
during the purge phase, a purge gas is produced,
and where the recycle gas from the PSA is composed of the waste stream compressed to the pressure P and/or of the purge gas compressed to the pressure P. According to this PSA process, in a first adsorption phase, the hydrogen-rich gaseous effluent resulting from R2 is brought into contact with a first adsorbent bed of the PSA and, in a second phase, it is the mixture of the other hydrogen-poor effluent(s) from the units R1 and R2 and of the recycle gas from the PSA which is brought into contact with this first adsorbent assembly. The recycle gas can be composed of two gases, alone or as a mixture: the waste gas resulting from the PSA, which has been compressed, and the purge gas resulting from the PSA, which has been compressed. Preferably, it is the purge gas and not the waste gas. The waste gas results from the final stage of the depressurization phase of the PSA and is partly compressed by the compressor incorporated in the PSA of the CPSA treatment device, whereas the purge gas results from the purge phase of the PSA and is partly compressed by this same compressor incorporated in the PSA before being used as recycle gas. These two gases both comprise hydrogen and essentially impurities. Once compressed, they are mixed with the hydrogen-poor effluents resulting from R1 and/or R2. This mixing can be carried out in various ways depending on the pressure values of the hydrogen-poor effluents resulting from R1 and R2. The hydrogen-poor effluent or effluents exhibiting very low pressures can be mixed with the waste gas or with the purge gas and then this mixture can be compressed by the compressor incorporated in the PSA up to the pressure P. If a hydrogen-poor effluent exhibits a pressure of greater than P, the compression of the other hydrogen-poor effluents may be avoided; in this case, only the waste gas or the purge gas is compressed to form the recycle gas. The introduction into the bed of the adsorbent of all these mixed gases at the pressure P allows them to be reprocessed. During the adsorption phase, the gaseous effluents are introduced in the bottom part of the bed in the “cocurrent” direction. During this contacting stage, the most adsorbable compounds, other than H₂, are adsorbed on the adsorbent and a gas comprising essentially hydrogen is produced at the pressure P reduced by approximately 1 bar of loss of head. During this stage, the hydrogen produced generally has a purity of greater than at least 99 mol %, preferably of greater than at least 99.5 mol %. This hydrogen can thus be used in another hydrogenation reaction unit, such as R3.

In order to obtain efficient purification, the adsorbent of the beds of the PSA has in particular to make possible the adsorption and the desorption of the impurities. The adsorbent bed is generally composed of a mixture of several adsorbents, said mixture comprising, for example, at least two adsorbents chosen from: active charcoals, silica gels, aluminas or molecular sieves. Preferably, the silica gels should exhibit a pore volume of between 0.4 and 0.8 cm³/g and a specific surface of greater than 600 m²/g. Preferably, the aluminas exhibit a pore volume of greater than 0.2 cm³/g and a specific surface of greater than 220 m²/g. The zeolites preferably have a pore size of less than 4.2 Å, have an Si/Al molar ratio of less than 5 and comprise Na and K. The active charcoals preferably exhibit a specific surface of greater than 800 m²/g and a micropore size of between 8 and 20 Å. According to a preferred form, each adsorbent bed of the PSA is composed of at least three layers of adsorbents of different natures. Each adsorbent bed of the PSA can comprise: in the bottom part, a protective layer composed of alumina and/or of silica gel surmounted by a layer of active charcoal and/or of carbon molecular sieve and optionally, in the top part, by a layer of molecular sieve. The proportions vary according to the nature of the gas mixture to be treated (in particular according to its percentages of CH₄ and of C₃₊ hydrocarbons) . For example, an anhydrous gas mixture comprising 75 mol % of H₂, 5 mol % of C₃₊ and 20 mol % of light hydrocarbons (C₁-C₂), of CO and of N₂ can be treated with an adsorption unit having beds comprising at least 10% by volume of alumina and 15% by volume of silica gel in the bottom bed, the remainder being obtained with active charcoal.

During the depressurization phase of the PSA, the waste gas is produced. The waste gas can be produced by countercurrentwise depressurization initiated at a pressure of less than P. This waste gas comprises the impurities and exhibits a lower hydrogen concentration than all the effluents resulting from R1 and R2. This waste gas can be discharged from the process and incinerated or reused as recycle gas in the CPSA as indicated above.

The low pressure of the cycle being reached, a purge phase is carried out in order to complete the regeneration of the adsorber. During the purge phase, a gas is introduced countercurrentwise into the adsorber and a purge gas is produced. The gas introduced countercurrentwise into the adsorber during the purge phase is a gas stream resulting from one of the stages of the depressurization phase. The purge gas is generally used as recycle gas after repressurization.

During the repressurization phase, the pressure of the adsorber is increased by countercurrentwise introduction of a gas stream comprising hydrogen, such as the gas produced during the various stages of the depressurization phase.

The use of the pressure swing adsorption unit in combination with an incorporated compressor (CPSA) exhibits the advantage of making possible the simultaneous treatment of all the effluents comprising hydrogen and of achieving better hydrogen recovery yields than if each stream had been treated separately by a pressure swing adsorption unit. In addition, due to the feeding of the CPSA by two separate effluents, it is possible to maintain uniform production of hydrogen for the third reaction unit. This is because the two units R1 and R2 can complement one another according to the effluents which they produce.

Generally, only parts of the effluents resulting from R1 and R2 are treated.

The invention can be implemented by a combination of the various units R1, R2 and R3 which may be found on the same site. Thus, the invention relates particularly to the case where the unit R1 is the unit for the hydrogenation of benzene of the synthesis of cyclohexane, the unit R2 is the unit for the hydrogenation of phenol or the synthesis of ε-caprolactum and R3 is the unit for the synthesis of a hydroxylamine, the hydrogenation of phenol and the synthesis of the hydroxylamine being two stages in the synthesis of caprolactam. The invention can be implemented with several R1 units and one R2 unit. The invention thus relates to the case where there exist two reaction units R1, one being a unit for the hydrodealkylation of toluene and the other a unit for the production of cyclohexane, and the unit R2 is a unit for the hydrodisproportionation of xylenes or toluene.

FIG. 1 illustrates a specific implementation of the process according to the invention. Two reaction units R1 and R2 in which hydrogen is employed are present on the petrochemical site illustrated. They are fed with hydrogen 2, 3 by a general and pure source of hydrogen 1. Subsequent to the reactions carried out in the units R1 and R2:
R2 produces two hydrogen-comprising effluents: the effluent 6, which is rich in hydrogen and exhibits a pressure P, and the effluent 7, which is depleted in hydrogen and exhibits a pressure which is less than P,
R1 produces two effluents comprising hydrogen: the effluent 5, which is depleted in hydrogen and exhibits a pressure P, and the effluent 4, which is depleted in hydrogen and exhibits a pressure which is less than P.

These four hydrogen-comprising effluents are treated by the separation unit U, which is a combination of a PSA and of a compressor. The hydrogen-rich effluent 6 is introduced at the head of the PSA and the impurities present therein are removed. The purge gas 10 from the PSA is mixed with the effluents 7 and 4, which are depleted in hydrogen and exhibit a pressure which is less than P. The mixture of these three effluents 10, 4 and 7 is optionally compressed by the compressor of the unit U until the pressure P is reached and the compressed mixture is mixed with the effluent 5 from the unit R1 so that the mixture of the effluents 4, 5 and 7 and of the purge gas 10 exhibits a pressure P and is treated by the PSA during one of the stages of the adsorption phase. The PSA produces the stream 9, which exhibits a greater hydrogen concentration than the effluent 6 and a pressure close to P. This stream 9 is used in a reaction unit R3, with or without a contribution from the source of high purity hydrogen 1. The PSA also produces a waste stream 8 of low pressure comprising the impurities from the various hydrogen-comprising effluents from the reaction units R1 and R2.

FIG. 2 illustrates a specific implementation of the alternative form of the process according to the invention. Three reaction units R11, R12 and R2 in which hydrogen is employed are present on the petrochemical site illustrated. R11 and R12 are equivalent: they are fed by a hydrogen-rich source and produce hydrogen-comprising effluents which feed the unit U. R11 is fed with hydrogen 21 by a general and pure source of hydrogen 1. R12 is fed with hydrogen 22 also by the source 1. The general source 1 can also feed the reaction unit R2. Subsequent to the reactions carried out in the units R11, R12 and R2:
R2 produces a hydrogen-comprising effluent: it is the effluent 6, which is rich in hydrogen and exhibits an initial pressure P,
R11 produces two hydrogen-comprising effluents: the effluent 51, which is depleted in hydrogen and exhibits a pressure which is greater than or equal to P, and the effluent 41, which is depleted in hydrogen and exhibits a pressure which is less than P,
R12 produces two hydrogen-comprising effluents: the effluent 52, which is depleted in hydrogen and exhibits a pressure which is greater than or equal to P, and the effluent 42, which is depleted in hydrogen and exhibits a pressure which is less than P.

These six hydrogen-comprising effluents are treated by the separation unit U, which is a combination of the PSA and of a compressor. The effluent 6 is introduced at the head of PSA and the impurities present therein are removed. The purge gas 10 from the PSA is mixed with the effluents 41 and 42 which are depleted in hydrogen. The mixture of these effluents 10, 41 and 42 can optionally be compressed by the compressor of the unit U until a pressure P is reached, by allowing this mixture, compressed and combined with the effluents 51 and 52 from the units R11 and R12, to exhibit a pressure P. According to one alternative form, at least one of the effluents 51 and/or 52 may exhibit a pressure which is greater than P; in this case, the use of the compressor may prove to be unnecessary if the simple mixing of the effluents 41, 42, 51 and 52 and of the purge gas 10 makes it possible to directly obtain a mixture at the pressure P. This mixture of the effluents, 41, 42, 51 and 52 and of the purge gas 10 at the pressure P is treated by the PSA during its adsorption phase. The PSA produces the stream 9, which exhibits a greater hydrogen concentration than the effluent 6 and a pressure close to P. This stream 9 is recycled in the reaction unit R2, with or without the contribution from the source of high purity hydrogen 1. The PSA also produces a waste stream 8 of low pressure comprising the impurities from the various hydrogen-comprising effluents from the reaction units R11, R12 and R2. According to the invention, it is not essential to have two R1 units. Depending on the petrochemical site studied, a single R1 unit or more than two R1 units may be employed in the process according to the invention. By the use of a device as defined above, it is possible for the operator of the petrochemical site on which the reaction units R1, R2 and R3 are located to improve the quality of the hydrogen-comprising gas used by the various units and to reduce his consumption of makeup hydrogen since it is no longer necessary to bleed (bleed 11 in FIG. 2), the hydrogen molecules being recovered from this bleeding operation. The process according to the invention can also allow the operator to debottleneck some of these reaction units if he continues to introduce a hydrogen-comprising makeup gas on his site simultaneously with the use of the process according to the invention.

There are a number of advantages to the invention in comparison with the existing solutions. First of all, it makes it possible to recover in value several hydrogen-comprising gases at the outlet of hydrogenation reaction units, whereas these gases are generally used as fuels. Subsequently, by virtue of the present invention, it is possible to purify a hydrogen-comprising recycle gas with the following advantages:
the hydrogen yield obtained during this purification of the recycle gas can exceed 100% (this hydrogen yield corresponding to the ratio of the hydrogen flow rate of the stream (9) resulting from the first outlet of the unit U to the hydrogen flow rate of the hydrogen-rich gaseous effluent (6),
the bleed (11) can be dispensed with,
the unit can be debottlenecked or the properties of its products can be improved. In addition, the contribution of “fresh” hydrogen is significantly reduced. Furthermore, the invention makes it possible to treat a gas mixture of high hydrogen purity, a gas mixture at high pressure of moderate hydrogen purity and a low-pressure gas mixture of low hydrogen purity in the same pressure swing adsorption cycle. Finally, the process according to the invention produces a waste stream at the pressure of the fuel gas system of the complex which can thus be discharged to this system.

### EXAMPLES

### Example 1

### Synthesis of ε-caprolactam by hydrogenation of phenol (Site for the Production of Nylons)

The scheme illustrated by FIG. 1 is applied to various stages of the process for the manufacture of ε-caprolactam by hydrogenation of phenol, the unit R1 being a unit for the hydrogenation of benzene of the synthesis of cyclohexane, the unit R2 being a unit for the hydrogenation of phenol of the synthesis of ε-caprolactam and the unit R3 being a unit for the synthesis of “the hydroxylamine”. These units can be found on the same site for the production of nylons.

The characteristics of the various effluents are summarized in table 1 below.

TABLE 1

| | Effluents from R1 | | Effluents from R2 | |
|---|---|---|---|---|
| | Effluent 4 | Effluent 5 | Effluent 6 | Effluent 7 |
| Flow rate (Nm³/h) | 1000 | 1500 | 5000 | 0 |
| P (bar) | 10 | 20 | 10 | 0 |
| % H₂ by volume | 30 | 70 | 97 | 0 |

The characteristics of the various effluents introduced and resulting from the purification unit U comprising the PSA and the compressor are summarized in table 2 below.

TABLE 2

| | | Mixture | Enriched | Waste |
|---|---|---|---|---|
| | Effluent 6 | 11 | effluent 9 | stream 8 |
| Flow rate of pure | 4850 | 1350 | 5600 | 600 |
| H₂ (Nm³/h) | | | | |
| % H₂ by volume | 97 | 54 | 99.99 | 31.6 |
| P (bar) | 10 | 3 | 10 | 3 |

The process according to the invention makes it possible to reduce the losses of hydrogen towards the fuel system of the site. Without the invention, the operation of the three units results in a loss of 6200 Nm³/h via bleeding operations. The installation of a conventional PSA for treating the hydrogen-rich effluent 6 makes it possible to reduce this loss to approximately 1850 Nm³/h. By virtue of the invention, it is possible to reduce this hydrogen loss to 600 Nm³/h. Consequently, the consumption of high purity makeup hydrogen (1) necessary for the operation of the unit R3 is reduced by 45%, changing from 12 500 to 6900 Nm³/h.

### Example 2

### Site for the Production of an Aromatic Complex

The scheme illustrated by FIG. 2 is implemented with two reaction units R1, referred to as R11 and R12, one being a unit for the hydrodealkylation of toluene and the other being a unit for the production of cyclohexane, and the unit R2 being a unit for the hydrodisproportionation of xylenes or toluene. These units can be found on the same site during the production of aromatic bases for the manufacture of polyesters, for example.

The unit R2 for the hydrodisproportionation of xylenes or toluene produces a hydrogen-comprising effluent with a hydrogen purity of close to 80% by volume. The invention makes it possible to purify this effluent and to recycle it towards the unit R2.

The characteristics of the various effluents are summarized in table 3 below.

TABLE 3

| | | | | | Effluent |
|---|---|---|---|---|---|
| | Effluents from R11 | | Effluents from R12 | | from R2 |
| | Effluent | Effluent | Effluent | Effluent | Effluent |
| | 51 | 41 | 52 | 42 | 6 |
| Flow rate | 5000 | 0 | 1500 | 1000 | 40 000 |
| (Nm³/h) | | | | | |
| P (bar) | 30 | 0 | 25 | 10 | 25 |
| % H₂ by | 55 | 0 | 70 | 30 | 80 |
| volume | | | | | |

The characteristics of the various effluents introduced and resulting from the purification unit U comprising the PSA and the compressor are summarized in table 4 below.

TABLE 4

| | | Mixture | Enriched | Waste |
|---|---|---|---|---|
| | Effluent 6 | 11 | effluent 9 | stream 8 |
| Flow rate | 32 000 | 4100 | 32 500 | 3600 |
| of pure H₂ | | | | |
| (Nm³/h) | | | | |
| % H₂ by | 80 | 55 | 99.9 | 24.7 |
| volume | | | | |
| P (bar) | 25 | 5 | 24 | 5 |

The process according to the invention makes it possible to increase the hydrogen partial pressure by 15 to 20% in the hydrodisproportionation reaction unit R2. It is thus possible to debottleneck this unit (increase in the capacity for the treatment of isomerized hydrocarbons). It is also possible to reduce the cracking reactions and to improve the selectivity for isomerized products at the same charging rate.

Finally, by the use of the invention, it is possible to dispense with all the bleeding of gas and the contribution of makeup hydrogen is extremely limited. Thus, dispensing with the bleeding operation makes it possible to achieve a saving of 1300 Nm³/h and the reduction in the make-up makes it possible to achieve a saving of 1500 Nm³/h.

1-10. (canceled)

11. A process for recovering in value the hydrogen-based gaseous effluents resulting from at least two reaction units R1 and R2 in which hydrogen is consumed, the unit R2 producing a hydrogen-rich gaseous effluent (6) at a pressure P and optionally a hydrogen-poor gaseous effluent (7) and the unit R1 producing at least one hydrogen-poor gaseous effluent (4, 5), characterized in that the following stages are carried out:
a) during stage a), all the hydrogen-poor gaseous effluents (5, 4, 7) resulting from R1 and optionally from R2 are mixed so that the mixture obtained exhibits a pressure P;
b) during stage b), the mixture of all the hydrogen-poor gaseous effluents (5, 4, 7) resulting from R1 and optionally from R2, adjusted to the pressure P during stage a), is treated in a gas separation unit U fed with the hydrogen-rich gaseous effluent (6) resulting from the unit R2 so as to provide, at a first outlet, an enriched stream (9) exhibiting a greater hydrogen concentration than that of the hydrogen-rich gaseous effluent (6) resulting from the unit R2 and, at a second outlet, a waste stream (10); and
c) during stage c), the enriched stream (9) resulting from the first outlet of the unit U is reinjected into a reaction unit R3 in which hydrogen is consumed.

12. The process as claimed in claim 11 characterized in that the hydrogen-rich effluent resulting from the unit R2 (6) exhibits a pressure of at least 5 bar.

13. The process as claimed in claim 11, characterized in that the hydrogen-rich effluent resulting from the unit R2 (6) exhibits a pressure of at least 15 bar.

14. The process as claimed in claim 11, characterized in that the hydrogen-rich effluent resulting from the unit R2 (6) exhibits a hydrogen concentration of between 50 and 99% by volume.

15. The process as claimed in claim 11, characterized in that the hydrogen-poor gaseous effluents (4, 5, 7) resulting from R1 and optionally from R2 exhibit a hydrogen concentration which is lower by least 10% with respect to the value of the hydrogen concentration of the hydrogen-rich effluent.

16. The process as claimed in claim 11, characterized in that the reaction unit R3 in which hydrogen is consumed is the reaction unit R2.

17. The process as claimed in claim 11, characterized in that the gas separation unit (U) is of the adsorption type.

18. The process as claimed in claim 17, characterized in that the gas separation unit (U) is a pressure swing adsorption (PSA) unit in combination with an incorporated compressor in which use is made, for each adsorber of the unit, of a pressure swing cycle comprising a sequence of phases which define adsorption, depressurization, purge and repressurization phases, such that:
a) during the adsorption phase:
1) during a first stage, the hydrogen-rich gaseous effluent (6) exhibiting a pressure P resulting from the unit R2 is brought into contact with the bed of the adsorber; and
2) during a second stage, the mixture with a pressure P composed:
i) on the one hand, of the mixture of all the hydrogen-poor gaseous effluents (5, 4, 7) resulting from R1 and optionally from R2 adjusted to the pressure P during stage a); and
ii) on the other hand, of the recycle gas from the PSA,
is introduced into contact with the bed of the adsorber,
so as to adsorb the compounds other than hydrogen and to produce, at the head of the bed of the adsorber, the enriched stream exhibiting a greater hydrogen concentration than that of the hydrogen-rich gaseous effluent (6) resulting from the unit R2;
b) during the depressurization phase, the waste stream (10) from the PSA is produced;
c) during the purge phase, a purge gas is produced; and
d) and where the recycle gas from the PSA is either the waste stream (10) compressed to the pressure P or the purge gas compressed to the pressure P.

19. The process as claimed in claim 11, characterized in that the unit R1 is the unit for the hydrogenation of benzene of the synthesis of cyclohexane, the unit R2 is the unit for the hydrogenation of phenol of the synthesis of ε-caprolactam and R3 is the unit for the synthesis of a hydroxylamine.

20. The process as claimed in claim 16, characterized in that it comprises two reaction units R1, one being a unit for the hydrodealkylation of toluene of benzene and the other a unit for the production of cyclohexane, and the unit R2 is a unit for the hydrodisproportionation of xylenes or toluene.
